(19) United States
(12) Patent Application Publication
(10) Pub. No.: US 2025/0228787 A1
(43) Pub. Date: Jul. 17, 2025

(54) BIOLOGICAL MATERIAL FOR DELIVERING SPECIFIC CELL-TARGETED METABOLIC SYSTEM, PREPARATION METHOD THEREFOR, AND APPLICATION THEREOF

(71) Applicant: ZHEJIANG UNIVERSITY, Hangzhou (CN)

(72) Inventors: Xianfeng LIN, Hangzhou (CN); Pengfei CHEN, Hangzhou (CN); Chenhui GU, Hangzhou (CN)

(21) Appl. No.: 19/060,736

(22) Filed: Feb. 23, 2025

Related U.S. Application Data
(63) Continuation of application No. PCT/CN2022/139153, filed on Dec. 14, 2022.

(30) Foreign Application Priority Data
Aug. 22, 2022 (CN) 202211004654.2

(51) Int. Cl. A61K 9/50

(52) U.S. Cl. CPC A61K 9/5068; A61K 9/5089

(57) ABSTRACT

Disclosed are a biological material for delivering a specific cell-targeted metabolic system, a preparation method therefor, and an application thereof. The biological material delivers the specific metabolic system into a specific cell by membrane fusion on the basis of cell membrane coating, so that the systematic metabolic regulation effect on the specific cell is achieved, and a new thought is provided for the preparation and application of the biological material designed for systematic cell metabolism regulation strategies.

### CROSS-REFERENCE TO RELATED APPLICATIONS

This application is a continuation of International Patent Application No. PCT/CN2022/139153 with a filing date of Dec. 14, 2022, designating the United States, now pending, and further claims priority to Chinese Patent Application No. 202211004654.2 with a filing date of Aug. 22, 2022. The content of the aforementioned applications, including any intervening amendments thereto, are incorporated herein by reference.

### TECHNICAL FIELD

The present invention relates to a biological material, and particularly to a biological material for delivering a specific cell-targeted metabolic system, a preparation method therefor, and an application thereof.

### BACKGROUND OF THE PRESENT INVENTION

The metabolic homeostasis of cells is very important for exerting a normal function of cells. Under pathological conditions such as inflammation and aging, a metabolic pattern of cells may change systematically. Although the research on a molecular mechanism of metabolic pathway of cells under these pathological conditions has been in-depth, current regulation modes often aim at individual links or molecules, and due to the existence of metabolic bypass and heterogeneity of cells, regulation modes aiming at single factors cannot achieve ideal effects at present, and there is still a lack of research and method to regulate cell metabolism from a systematic level.

Metabolism in cells is divided into two parts, which are namely energy metabolism and substance metabolism. The energy metabolism mainly refers to the synthesis and decomposition of high-energy molecules such as ATP, while the substance metabolism mainly refers to the synthesis and decomposition of biomolecules (proteins, lipids, sugars, and the like) based on energy supply such as ATP and NADPH. These complex metabolic processes mainly occur in nucleuses, cytoplasmic matrices, mitochondria, chloroplasts, endoplasmic reticula and Golgi apparatuses, and these cell components may be used as material sources for systematic metabolic regulation of cells. Meanwhile, in order to transfer these metabolic systems into specific cells, effective delivery is needed, and cell membrane coating is an ideal membrane-fusion delivery mode.

Firstly, at present, inventions aiming at the above metabolic systems mainly focus on drug therapy targeting the metabolic system itself. The patent with the application number CN201610371113.1 discloses a mitochondrion-targeted nano-drug delivery system, a preparation method therefor, and an application thereof, wherein TPP is selectively located in mitochondria, and a chemotherapy drug is released through ROS response. The patent with the application number CN202011399804.5 discloses construction and application of an endoplasmic reticulum-targeted nano-drug delivery system, wherein a drug is delivered through an endoplasmic reticulum-targeted nano-compound constructed by modifying a sulfamide or sulfonylurea compound with endoplasmic reticulum tropism into a nano-carrier. Secondly, at present, the application of cell membrane is mainly limited to the delivery of micromolecules, proteins, genes, and the like. The patent with the application number CN201810588367.8 discloses a preparation method of an anti-tumor therapeutic agent loaded by an autophagy-imitated immune cell, wherein the anti-tumor therapeutic agent is encapsulated with a cell membrane having an apoptosis group, an application thereof is limited to a tumor cell membrane, and internally encapsulated substances are limited to drug nanoparticles. Such patents fail to design the delivery of whole metabolic system to a target cell, so as to realize the systematic metabolic regulation function of a specific cell. Therefore, it is necessary to prepare a biological material for delivering a specific cell-targeted metabolic system, wherein membrane fusion delivery is carried out through a cell membrane, and the metabolic system is delivered to the specific cell, so as to realize systematic metabolic regulation, and effectively maintain a metabolic state of the cell.

### SUMMARY OF THE PRESENT INVENTION

Aiming at the defects in the prior art, the present invention provides a biological material for delivering a specific cell-targeted metabolic system, a preparation method therefor, and an application thereof. The biological material delivers the specific metabolic system into a specific cell by membrane fusion on the basis of cell membrane coating, so that the systematic metabolic regulation effect on the specific cell is achieved.

In order to achieve the objective above, the present invention provides the following technical solutions.

The present invention provides a biological material for delivering a specific cell-targeted metabolic system, wherein the biological material is composed of a cell membrane of a target cell for delivery and a metabolic system component encapsulated inside.

Preferably, the cell membrane of the target cell for delivery comprises a cell membrane of a motor system-related cell, a circulatory system-related cell, a digestive system-related cell, a urinary system-related cell, a nervous system-related cell, a germ cell, an endocrine cell and a tumor cell; the cell membrane of the motor system-related cell comprises a cell membrane of a chondrocyte, a myocyte, an osteoblast, an osteoclast and a mesenchymal stem cells the cell membrane of the circulatory system-related cell comprises a cell membrane of a hematopoietic stem cell, a monocyte, a granulocyte, a macrophage, a B lymphocyte, a T lymphocyte, an erythrocyte, a platelet, a myocardial cell and a vascular endothelial cell; the cell membrane of the digestive system-related cell comprises a cell membrane of a hepatocyte, a gastrointestinal epithelial cell, a goblet cell and an islet cell; the cell membrane of the urinary system-related cell comprises a cell membrane of a respiratory system cell such as an alveolar cell and a tracheal epithelial cell, a glomerular endothelial cell and a renal tubular epithelial cell; and the cell membrane of the nervous system-related cell comprises a cell membrane of a neuronal cell, an astrocyte, an oligodendrocyte and a microglial cell.

Preferably, the cell membrane of the target cell for delivery is a cell membrane of a chondrocyte, a myocyte, a monocyte, a macrophage, an endothelial cell and an epithelial cell.

Preferably, the cell membrane of the target cell for delivery is a cell membrane of a chondrocyte having definite differentiation characteristics and targeting by intra-articular injection to avoid systemic influence.

Preferably, the metabolic system comprises an entirety and partial contents of a nucleus, a cytoplasmic matrix, a mitochondrion, a chloroplast, an endoplasmic reticulum and a Golgi apparatus.

Preferably, the metabolic system is content component-containing thylakoid vesicle in chloroplast.

A preparation method for the biological material for delivering the specific cell-targeted metabolic system is provided, which comprises the following steps of:
extracting metabolic system vesicle to be delivered for granulation, extracting the cell membrane of the target cell for delivery, and encapsulating the metabolic system into the cell membrane, so as to obtain cell membrane vesicle encapsulating the metabolic system component.

Preferably, the metabolic system vesicle to be delivered is thylakoid vesicle, which is extracted in the following mode:
a plant green leaf material and a cold buffer A are mixed by a blender in a ratio of 1 g:1 mL; the obtained solution is pressed to pass through a fine mesh cotton fabric, and a filtrate is centrifuged at 3000 g for 10 minutes; a precipitate is resuspended in a buffer B; the solution is added to 80/40% Percoll gradient solution; a part containing a green layer is collected to obtain a thylakoid; ingredients of the buffer A are sorbitol, HEPES-KOH with pH of 7.6, MgCl₂ and 0.1% BSA; and ingredients of the buffer Bare sorbitol, HEPES-KOH with pH of 7.6, MgCl₂, EDTA and L-sodium ascorbate.

Preferably, a granulating process is implemented by an ultrasonic extrusion method, which is carried out in the following mode:
the metabolic system vesicle to be delivered is subjected to an ultrasonic treatment in a bath ultrasonic instrument, and repeatedly extruded by a polycarbonate porous membrane; then the solution is centrifuged at 3000 g for 10 minutes; and a precipitate is resuspended in a buffer D, wherein ingredients of the buffer D are HEPES-KOH, MgCl₂ and sodium ascorbate.

Preferably, conditions of the ultrasonic treatment of the ultrasonic instrument comprise: a No. 2 amplitude-change pole, 20% to 60% power, turning on for 2 seconds, turning off for 3 seconds, and working for 2 minutes.

Preferably, a pore size of the polycarbonate membrane above is 50 nm to 200 nm.

Preferably, the extraction of the cell membrane of the target cell for delivery is carried out in the following mode:
the cell is collected and resuspended in a buffer E at 4° C., repeatedly whipped with an insulin needle for 20 times to lyse the cell, and mixed with a 1 M sucrose buffer E solution in a ratio of 3:1 into a 0.25 M sucrose buffer E solution, the solution is centrifuged at 2000 g for 10 minutes, a supernatant is taken and centrifuged at 3000 g for 30 minutes, and a precipitate is the cell membrane; ingredients of the buffer E are mannitol, sucrose, Tris, MgCl₂, KCl, PMSF, a protease inhibitor without EDTA, DNase and RNase, and a pH value of the buffer E is 7.4; and a concentration of the Tris is 5 mM to 30 mM, and a concentration of the MgCl₂ is 1 mM to 20 mM.

Preferably, the concentration of the Tris is 10 mM.

Preferably, the concentration of the MgCl₂ is 1 mM.

Preferably, an encapsulating process is carried out in the following mode:
the metabolic system component is loaded into the cell membrane vesicle by methods comprising microporous extrusion, ultrasonic hydration and a microfluidic technology.

Preferably, the encapsulating process is implemented by a filter-membrane microporous gradient extrusion method, and pore sizes of filter-membrane micropores are 1000 nm, 400 nm and 200 nm gradiently.

An application of a biological material for delivering a specific cell-targeted metabolic system is provided, wherein the biological material for delivering the specific cell-targeted metabolic system is applied to a target cell, so that the target cell ingests the biological material and internalizes the metabolic system, thus realizing a specific function of the metabolic system in the cell.

Preferably, an applying process comprises adding the biological material for delivering the specific cell-targeted metabolic system into a culture medium in an in-vitro cell culture system, and conveying the biological material for delivering the specific cell-targeted metabolic system to a specific cell through local injection, intravenous injection and the like in in-vivo use.

The present invention has the beneficial effects as follows:
1) According to the biological material for delivering the specific cell-targeted metabolic system provided by the present invention, there are high targeting ability and high selectivity to the target cell.
2) According to the biological material for delivering the specific cell-targeted metabolic system provided by the present invention, there is an effective regulation effect on a key metabolic molecule of the target cell.
3) According to the biological material for delivering the specific cell-targeted metabolic system provided by the present invention, the adjustment of systemic metabolic state of the target cell is realized.
4) According to the biological material for delivering the specific cell-targeted metabolic system provided by the present invention, there is a significant therapeutic effect on diseases of cell metabolism disorder.

### DESCRIPTION OF THE DRAWINGS

In order to make the objectives, technical solutions and beneficial effects of the present invention clearer, the present invention provides the following drawings for description:

FIG. 1(a) and FIG. 1(b) show flow cytometry analysis of a biological material for delivering a chondrocyte-targeted thylakoid metabolic system in Embodiment 1 ingested by different cells in a mixed cell system, indicating that an uptake degree of chondrocyte is significantly higher than those of nucleus pulposus cell, fibroblast, muscle satellite cell and macrophage, so that the biological material for delivering the chondrocyte-targeted thylakoid metabolic system has high targeting ability and selectivity for the chondrocyte.

FIG. 2(a ) and FIG. 2(b) show flow cytometry analysis of biological materials for delivering thylakoid metabolic systems encapsulated with different cell membranes in Embodiment 1 ingested by chondrocytes, indicating that an uptake degree of chondrocyte to a biological material for delivering a thylakoid metabolic system encapsulated with a chondrocyte membrane is significantly higher than uptake degrees of chondrocyte to biological materials for delivering thylakoid metabolic systems encapsulated with cell membranes of nuclear nucleus cell, fibroblast, muscle satellite cell and macrophage, so that the chondrocyte has uptake tropism to the biological material for delivering the thylakoid metabolic system encapsulated with the chondrocyte membrane.

FIG. 3(a) and FIG. 3(b) show quantitative measurement of a key metabolic molecule ATP generated in chondrocyte by the biological material for delivering the chondrocyte-targeted thylakoid metabolic system in Embodiment 1, indicating that the thylakoid metabolic system can effectively increase an ATP content in chondrocyte.

FIG. 4(a ) and FIG. 4(b) show a fluoroscopic image of substance metabolism regulation in chondrocyte of the biological material for delivering the chondrocyte-targeted thylakoid metabolic system in Embodiment 1, indicating that the thylakoid metabolic system can effectively promote the synthesis and metabolism of substances in chondrocyte.

FIG. 5 shows combined transcriptomic and metabonomic analysis of systematic metabolism regulation in chondrocyte of the biological material for delivering the chondrocyte-targeted thylakoid metabolic system in Embodiment 1, indicating that the thylakoid metabolic system can effectively promote oxidative phosphorylation and substance synthesis of the chondrocyte, reduce glycolysis, and systematically stabilize a metabolic level of the chondrocyte.

FIG. 6(a ) to FIG. 6(d) show Safranine O-Fast Green staining of a therapeutic effect on osteoarthritis in mice of the biological material for delivering the chondrocyte-targeted thylakoid metabolic system in Embodiment 1, indicating that the thylakoid metabolic system can effectively inhibit the progression of osteoarthritis by systematically regulating the metabolism of the chondrocyte.

### DETAILED DESCRIPTION OF PREFERRED EMBODIMENTS

A biological material for delivering a specific cell-targeted metabolic system, a preparation method therefor, and an application thereof provided by the present invention are described in detail hereinafter with reference to embodiments, but the embodiments cannot be understood as limiting the scope of protection of the present invention.

The present invention provides a biological material for delivering a specific cell-targeted metabolic system, wherein the biological material is composed of a cell membrane of a specific cell and a metabolic system component encapsulated inside. The “cell membrane of the specific cell” refers to a cell membrane of a target cell for delivery, comprising a cell membrane of a motor system-related cell such as a chondrocyte, a myocyte, an osteoblast, an osteoclast and a mesenchymal stem cell; a circulatory system-related cell such as a hematopoietic stem cell, a monocyte, a granulocyte, a macrophage, a B lymphocyte, a T lymphocyte, an erythrocyte, a platelet, a myocardial cell and a vascular endothelial cell; a digestive system-related cell such as a hepatocyte, a gastrointestinal epithelial cell, a goblet cell and an islet cell; a urinary system-related cell such as, a respiratory system cell such as an alveolar cell and a tracheal epithelial cell, a glomerular endothelial cell and a renal tubular epithelial cell; a nervous system-related cell such as a neuronal cell, an astrocyte, an oligodendrocyte and a microglial cell; and a germ cell, an endocrine cell, a tumor cell, and the like. Preferably, the cell membrane of the specific cell is a cell membrane of a cell having definite differentiation characteristics such as a chondrocyte, a myocyte, a monocyte, a macrophage, an endothelial cell and an epithelial cell, and more preferably, the cell membrane of the specific cell is a cell membrane of a chondrocyte having definite differentiation characteristics and targeting by intra-articular injection to avoid systemic influence. The “metabolic system” comprises an entirety and partial contents of a nucleus, a cytoplasmic matrix, a mitochondrion, a chloroplast, an endoplasmic reticulum and a Golgi apparatus. Preferably, the metabolic system is the chloroplast, and more preferably, the metabolic system is content component-containing thylakoid vesicle in chloroplast.

A preparation method for the biological material for delivering the specific cell-targeted metabolic system provided by the present invention comprises the following steps.

In step 1, metabolic system vesicle to be delivered is preferably thylakoid vesicle, which is extracted in the following mode.

A plant green leaf material and a cold buffer A are mixed by a blender in a ratio of 1 g:1 mL. The obtained solution is pressed to pass through a fine mesh cotton fabric, and a filtrate is centrifuged at 3000 g for 10 minutes. A precipitate is gently resuspended in a buffer B. The solution is added to 80/40% Percoll gradient solution. A part containing a green layer is collected to obtain a thylakoid. Ingredients of the buffer A above are 330 mM sorbitol, 50 mM HEPES-KOH with pH of 7.6, 5 mM MgCl₂ and 0.1% BSA. Ingredients of the buffer B above are 300 mM sorbitol, 50 mM HEPES-KOH with pH of 7.6, 5 mM MgCl₂, 2 mM EDTA and 10 mM L-sodium ascorbate. A preparation method of the 80/40% Percoll gradient solution above is: 80% Percoll:80% v/v Percoll, 10 mM L-sodium ascorbate, 300 mM sucrose and 66 mM MOPS-KOH with pH of 7.6; and 40% Percoll:40% v/v Percoll, 10 mM L-sodium ascorbate, 300 mM sucrose and 25 mM MOPS-KOH with pH of 7.6.

In step 2, a granulating process is preferably implemented by an ultrasonic extrusion method, and is carried out in the following mode.

The metabolic system vesicle to be delivered is subjected to an ultrasonic treatment in a bath ultrasonic instrument (under conditions of a No. 2 amplitude-change pole, 20% to 60% power, turning on for 2 seconds, turning off for 3 seconds, and working for 2 minutes), and repeatedly extruded by a polycarbonate porous membrane with a pore size of 100 nm. Then, the solution is centrifuged at 3000 g for 10 minutes. A precipitate is resuspended in a buffer D. Ingredients of the buffer D above are 10 mM HEPES-KOH, 10 mM MgCl₂ and 10 mM L-sodium ascorbate.

In step 3, the extraction of the specific cell membrane is carried out in the following mode.

The cell is collected and resuspended in a buffer E at 4° C., repeatedly whipped with an insulin needle for 20 times to lyse the cell, and mixed with a 1 M sucrose buffer E solution in a ratio of 3:1 into a 0.25 M sucrose buffer E solution, the solution is centrifuged at 2000 g for 10 minutes, a supernatant is taken and centrifuged at 3000 g for 30 minutes, and a precipitate is the cell membrane. Ingredients of the buffer E above were mannitol, sucrose, Tris, MgCl₂, KCl, PMSF, a protease inhibitor without EDTA, DNase and RNase, and a pH value of the buffer E was 7.4. A concentration of the Tris is 5 mM to 30 mM, and more preferably, the concentration of the Tris is 10 mM. A concentration of the MgCl₂ is 1 mM to 20 mM, and more preferably, the concentration of the MgCl₂ is 1 mM.

In step 4, an encapsulating process is carried out in the following mode.

The metabolic system component is loaded into the cell membrane vesicle by methods comprising microporous extrusion, ultrasonic hydration and a microfluidic technology. Preferably, a filter-membrane microporous gradient extrusion method is adopted. More preferably, pore sizes of filter-membrane micropores are 1000 nm, 400 nm and 200 nm gradiently.

An application of a biological material for delivering a specific cell-targeted metabolic system provided by the present invention comprises application contents as follows.

The biological material for delivering the specific cell-targeted metabolic system is applied to a target cell, so that the target cell ingests the biological material and internalizes the metabolic system, thus realizing a specific function of the metabolic system in the cell. The “applying” process above comprises adding the biological material for delivering the specific cell-targeted metabolic system into a culture medium in an in-vitro cell culture, and delivering the biological material for delivering the specific cell-targeted metabolic system to a specific cell through local injection, intravenous injection and the like in in-vivo use.

The present invention may also adopt other metabolic system components, other specific cell membranes, and other encapsulating processes mentioned above, all of which can achieve the same technical effects.

### Embodiment 1. Encapsulation of Thylakoid With Chondrocyte Membrane by Filter Membrane Microporous Gradient Extrusion Method for Treating Osteoarthritis Through Intra-Articular Injection

In step 1, metabolic system thylakoid vesicle to be delivered was extracted in the following mode.

A plant green leaf material and a cold buffer A were mixed by a blender in a ratio of 1 g:1 mL. The obtained solution was pressed to pass through a fine mesh cotton fabric, and a filtrate was centrifuged at 3000 g for 10 minutes. A precipitate was gently resuspended in a buffer B. The solution was added to 80/40% Percoll gradient solution. A part containing a green layer was collected to obtain a thylakoid. Ingredients of the buffer A above were 330 mM sorbitol, 50 mM HEPES-KOH with pH of 7.6, 5 mM MgCl₂ and 0.1% BSA. Ingredients of the buffer B above were 300 mM sorbitol, 50 mM HEPES-KOH with pH of 7.6, 5 mM MgCl₂, 2 mM EDTA and 10 mM L-sodium ascorbate. A preparation method of the 80/40% Percoll gradient solution above was: 80% Percoll:80% v/v Percoll, 10 mM L-sodium ascorbate, 300 mM sucrose and 66 mM MOPS-KOH with pH of 7.6; and 40% Percoll:40% v/v Percoll, 10 mM L-sodium ascorbate, 300 mM sucrose and 25 mM MOPS-KOH with pH of 7.6.

In step 2, a granulating process was implemented by an ultrasonic extrusion method, and was carried out in the following mode.

The metabolic system vesicle to be delivered was subjected to an ultrasonic treatment in a bath ultrasonic instrument (under conditions of a No. 2 amplitude-change pole, 20% to 60% power, turning on for 2 seconds, turning off for 3 seconds, and working for 2 minutes), and repeatedly extruded by a polycarbonate porous membrane with a pore size of 100 nm. Then, the solution was centrifuged at 3000 g for 10 minutes. A precipitate was resuspended in a buffer D. Ingredients of the buffer D above were 10 mM HEPES-KOH, 10 mM MgCl₂ and 10 mM L-sodium ascorbate.

In step 3, the extraction of the chondrocyte membrane was carried out in the following mode.

The chondrocyte was collected and resuspended in a buffer E at 4° C., repeatedly whipped with an insulin needle for 20 times to lyse the cell, and mixed with a 1 M sucrose buffer E solution in a ratio of 3:1 into a 0.25 M sucrose buffer E solution, the solution was centrifuged at 2000 g for 10 minutes, a supernatant was taken and centrifuged at 3000 g for 30 minutes, and a precipitate was the cell membrane. Ingredients of the buffer E above were mannitol, sucrose, Tris, MgCl₂, KCl, PMSF, a protease inhibitor without EDTA, DNase and RNase, and a pH value of the buffer E was 7.4. A concentration of the Tris was 5 mM to 30 mM, and more preferably, the concentration of the Tris was 10 mM. A concentration of the MgCl₂ was 1 mM to 20 mM, and more preferably, the concentration of the MgCl₂ was 1 mM.

In step 4, an encapsulating process was carried out in the following mode.

A metabolic system component was loaded into the cell membrane vesicle, the encapsulating process was implemented by a filter-membrane microporous gradient extrusion method, and pore sizes of filter-membrane micropores were 1000 nm, 400 nm and 200 nm gradiently.

### Embodiment 2. Encapsulation of Mitochondrion With Hepatocyte Membrane by Filter Membrane Microporous Gradient Extrusion Method for Treating Acute Hepatitis Through Tail Intravenous Injection

In step 1, metabolic system mitochondrion vesicle to be delivered was extracted in the following mode.

The hepatocyte was collected and resuspended in a buffer E at 4° C., repeatedly whipped with an insulin needle for 20 times to lyse the cell, and mixed with a 1 M sucrose buffer E solution in a ratio of 3:1 into a 0.25 M sucrose buffer E solution. The solution was added to an Opti-prep gradient solution for centrifugation, a part containing a specific gravity gradient of mitochondria was collected to obtain a mitochondrion. Ingredients of the buffer E above were mannitol, sucrose, Tris, MgCl₂, KCl, PMSF, a protease inhibitor without EDTA, DNase and RNase, and a pH value of the buffer E was 7.4. A concentration of the Tris was 5 mM to 30 mM, and more preferably, the concentration of the Tris was 10 mM. A concentration of the MgCl₂ was 1 mM to 20 mM, and more preferably, the concentration of the MgCl₂ was 1 mM.

In step 2, a granulating process was implemented by an ultrasonic extrusion method, and was carried out in the following mode.

The metabolic system vesicle to be delivered was subjected to an ultrasonic treatment in a bath ultrasonic instrument (under conditions of a No. 2 amplitude-change pole, 20% to 60% power, turning on for 2 seconds, turning off for 3 seconds, and working for 2 minutes), and repeatedly extruded by a polycarbonate porous membrane with a pore size of 100 nm. Then, the solution was centrifuged at 3000 g for 10 minutes. A precipitate was resuspended in a buffer D. Ingredients of the buffer D above were 10 mM HEPES-KOH, 10 mM MgCl₂ and 10 mM L-sodium ascorbate.

In step 3, the extraction of the hepatocyte membrane was carried out in the following mode.

The hepatocyte was collected and resuspended in a buffer E at 4° C., repeatedly whipped with an insulin needle for 20 times to lyse the cell, and mixed with a 1 M sucrose buffer E solution in a ratio of 3:1 into a 0.25 M sucrose buffer E solution, the solution was centrifuged at 2000 g for 10 minutes, a supernatant was taken and centrifuged at 3000 g for 30 minutes, and a precipitate was the cell membrane. Ingredients of the buffer E above were mannitol, sucrose, Tris, MgCl₂, KCl, PMSF, a protease inhibitor without EDTA, DNase and RNase, and a pH value of the buffer E was 7.4. A concentration of the Tris was 5 mM to 30 mM, and more preferably, the concentration of the Tris was 10 mM. A concentration of the MgCl₂ was 1 mM to 20 mM, and more preferably, the concentration of the MgCl₂ was 1 mM.

In step 4, an encapsulating process was carried out in the following mode.

Metabolic system component-containing mitochondrion vesicle was loaded into the hepatocyte membrane vesicle, the encapsulating process was implemented by a filter-membrane microporous gradient extrusion method, and pore sizes of filter-membrane micropores were 1000 nm, 400 nm and 200 nm gradiently.

### Embodiment 3. Encapsulation of Osteoblast Cytoplasmic Matrix With Monocyte Membrane by Microfluidic Method

In step 1, a metabolic system osteoblast cytoplasmic matrix to be delivered was extracted in the following mode.

The osteoblast was collected and resuspended in a buffer E at 4° C., repeatedly whipped with an insulin needle for 20 times to lyse the cell, and mixed with a 1 M sucrose buffer E solution in a ratio of 3:1 into a 0.25 M sucrose buffer E solution. The solution was centrifuged at 2000 g for 10 minutes. A supernatant was centrifuged at 3000 g for 10 minutes. A supernatant was added with the same amount of ethanol and ¼ amount of chloroform, and mixed evenly. The mixture was centrifuged at 12000 rpm for 5 minutes, and an upper layer was discarded. The product was added with 500 ul of ethanol, mixed evenly in a translational manner, and centrifuged at 12000 rpm for 5 minutes, an upper layer was discarded, and a precipitate was resuspended to obtain a cytoplasmic protein. Ingredients of the buffer E above were mannitol, sucrose, Tris, MgCl₂, KCl, PMSF, a protease inhibitor without EDTA, DNase and RNase, and a pH value of the buffer E was 7.4. A concentration of the Tris was 5 mM to 30 mM, and more preferably, the concentration of the Tris was 10 mM. A concentration of the MgCl₂ was 1 mM to 20 mM, and more preferably, the concentration of the MgCl₂ was 1 mM.

In step 2, the extraction of the monocyte membrane was carried out in the following mode.

The monocytes was collected and resuspended in a buffer E at 4° C., and repeatedly whipped with an insulin needle for 20 times to lyse the cell, a cell homogenate and a 1 M sucrose buffer E solution were mixed in a ratio of 3:1 into a 0.25 M sucrose buffer E solution, the solution was centrifuged at 2000 g for 10 minutes, a supernatant was taken and centrifuged at 3000 g for 30 minutes, and a precipitate was the cell membrane. Ingredients of the buffer E above were mannitol, sucrose, Tris, MgCl₂, KCl, PMSF, a protease inhibitor without EDTA, DNase and RNase, and a pH value of the buffer E was 7.4. A concentration of the Tris was 10 mM, and a concentration of the MgCl₂ was 1 mM.

In step 3, an encapsulating process was carried out in the following mode.

The monocyte membrane was dissolved in ethanol, the osteoblast cytoplasmic matrix protein was dissolved in water, the osteoblast cytoplasmic matrix protein aqueous solution was encapsulated into a cell membrane-containing liquid flow through a microfluidic V-chip, and the ethanol was removed through dialysis, so as to prepare the monocyte membrane-encapsulated osteoblast cytoplasmic matrix protein.

### Flow Cytometry Analysis of Biological Material for Delivering Chondrocyte-Targeted Thylakoid Metabolic System in Embodiment 1 Ingested by Different Cells in Mixed Cell System

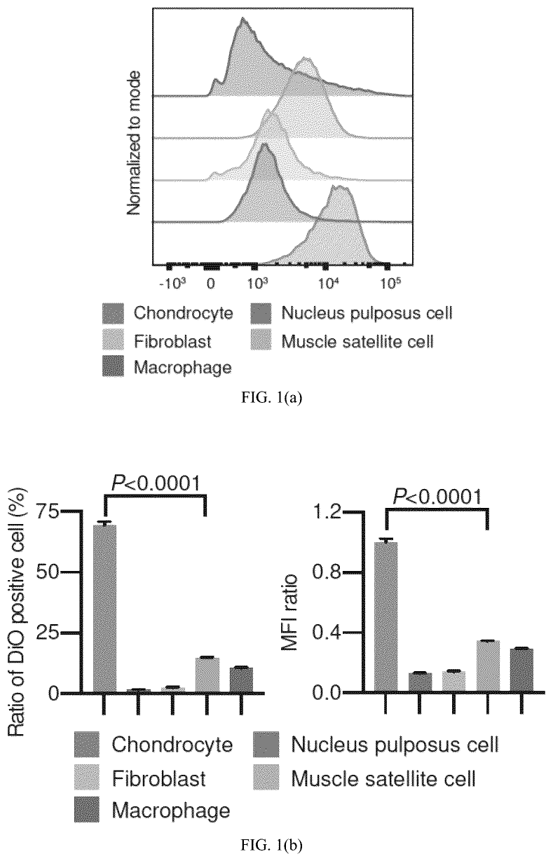

1. The chondrocyte, the nucleus pulposus cell, the fibroblast, the muscle satellite cell and the macrophage were mixed and cultured.
2. The biological material for delivering the chondrocyte-targeted thylakoid metabolic system was added into the mixed culture system.
3. The digestively acquisitive cell was subjected to flow cytometry analysis. It was indicated that an uptake degree of chondrocyte was significantly higher than those of nucleus pulposus cell, fibroblast, muscle satellite cell and macrophage, so that the biological material for delivering the chondrocyte-targeted thylakoid metabolic system had high targeting ability and selectivity for the chondrocyte, as shown in FIG. 1(a ) and FIG. 1(b).

### Flow Cytometry Analysis of Biological Materials for Delivering Thylakoid Metabolic Systems Encapsulated With Different Cell Membranes in Embodiment 1 Ingested by Chondrocyte

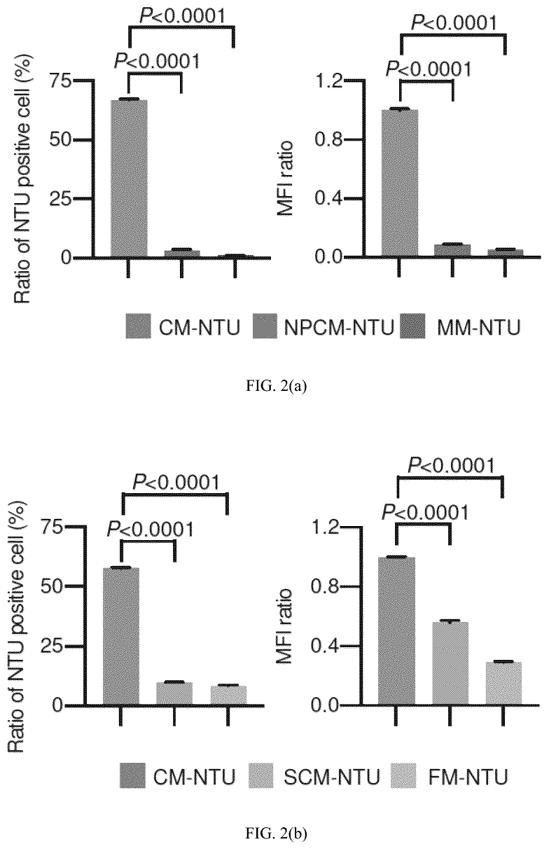

1. The cell membranes of the chondrocyte, the nucleus pulposus cell, the fibroblast, the muscle satellite cell and the macrophage were extracted respectively to encapsulate the metabolic system component-containing thylakoid.
2. The biological materials for delivering the thylakoid metabolic systems encapsulated with different cell membranes were added into the chondrocyte culture system.
3. The digestively acquisitive cell was subjected to flow cytometry analysis. It was indicated that an uptake degree of chondrocyte to a biological material for delivering a thylakoid metabolic system encapsulated with the chondrocyte membrane was significantly higher than uptake degrees of chondrocyte to biological materials for delivering thylakoid metabolic systems encapsulated with the cell membranes of the nuclear nucleus cell, the fibroblast, the muscle satellite cell and the macrophage, so that the chondrocyte had uptake tropism to the biological material for delivering the thylakoid metabolic system encapsulated with the chondrocyte membrane, as shown in FIG. 2(a ) and FIG. 2(b).

### Quantitative Measurement of Key Metabolic Molecule ATP Generated in Chondrocyte by Biological Material for Delivering Chondrocyte-Targeted Thylakoid Metabolic System in Embodiment 1

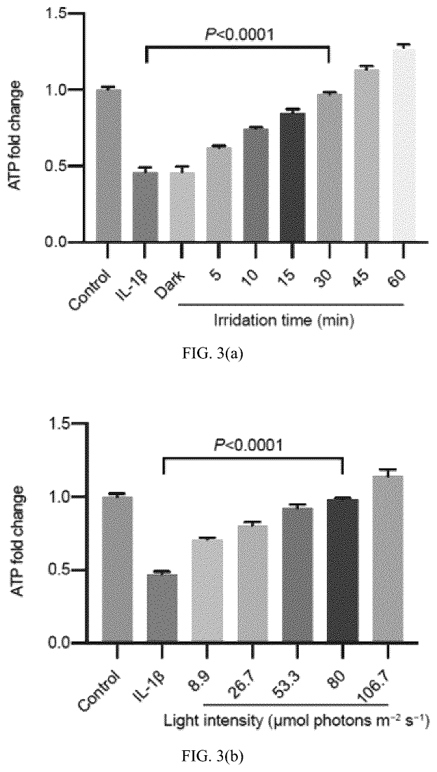

1. The biological material for delivering the chondrocyte-targeted thylakoid metabolic system was added into the chondrocyte culture system.
2. Different gradients of illumination time and intensity were applied to the culture system.
3. Results showed that the thylakoid metabolic system could effectively increase an ATP content in chondrocyte, as shown in FIG. 3(a ) and FIG. 3(b).

### Fluoroscopic Image of Substance Metabolism Regulation in Chondrocyte of Biological Material for Delivering Chondrocyte-Targeted Thylakoid Metabolic System in Embodiment 1

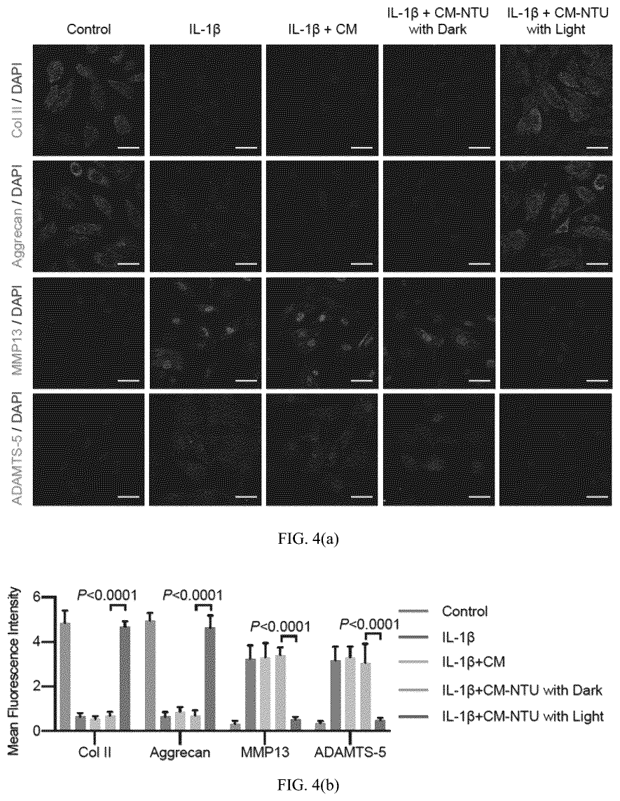

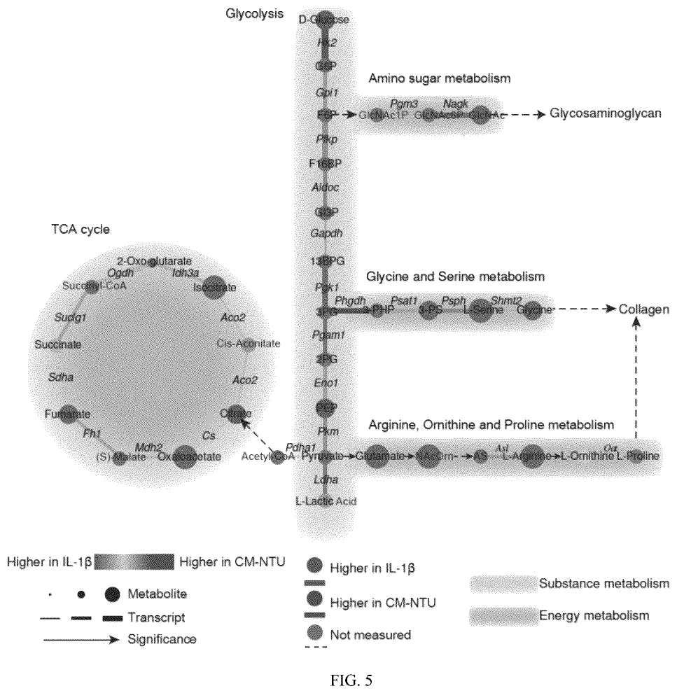

1. The biological material for delivering the chondrocyte-targeted thylakoid metabolic system was added into a culture system of chondrocyte and inflammatory chondrocyte.
2. After phototherapy, the cells was fixed, and added with a primary antibody and a fluorescent secondary antibody respectively, and indexes of anabolism and catabolism were observed.
3. It was indicated that the thylakoid metabolic system could effectively promote the synthesis and metabolism of substances in chondrocyte, as shown in FIG. 4(a) and FIG. 4(b).

### Combined Transcriptomic and Metabonomic Analysis of Systematic Metabolism Regulation in Chondrocyte of Biological Material for Delivering Chondrocyte-Targeted Thylakoid Metabolic System in Embodiment 1

1. The biological material for delivering the chondrocyte-targeted thylakoid metabolic system was added into the culture system of chondrocyte and inflammatory chondrocyte.
2. The chondrocyte was collected, and subjected to transcriptome and metabolome sequencing.
3. Sequencing results were subjected to combined analysis, and results showed that the thylakoid metabolic system could effectively promote oxidative phosphorylation and substance synthesis of the chondrocyte, reduce glycolysis, and systematically stabilize a metabolic level of the chondrocyte, as shown in FIG. 5.

### Safranine O-Fast Green Staining of Therapeutic Effect on Osteoarthritis in Mice of Biological Material for Delivering Chondrocyte-Targeted Thylakoid Metabolic System in Embodiment 1

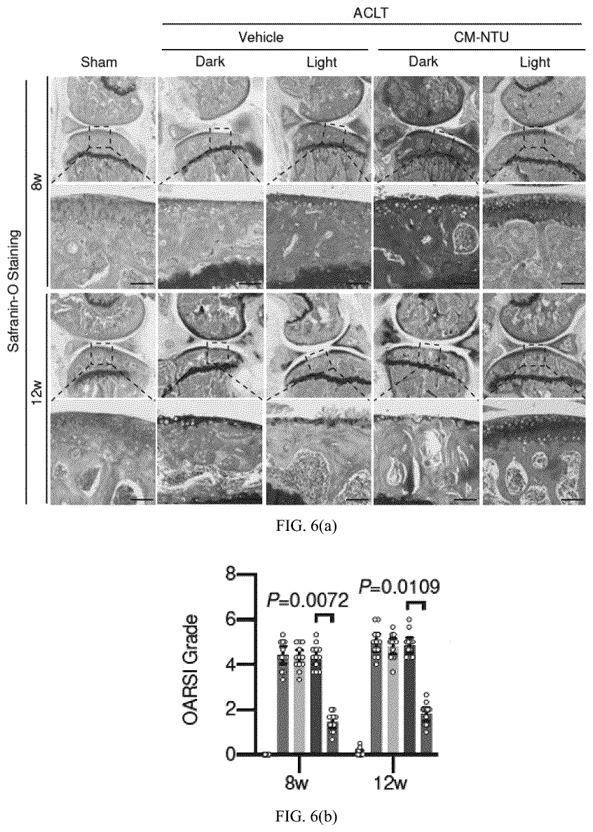

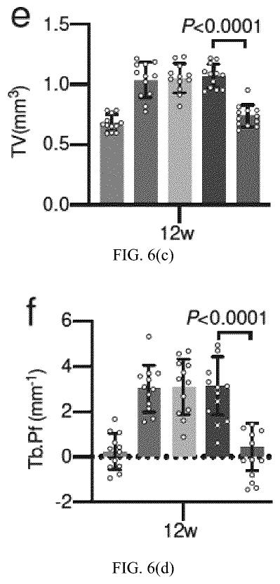

1. The biological material for delivering the chondrocyte-targeted thylakoid metabolic system was applied into an articular cavity of a mouse with arthritis through intra-articular injection.
2. A joint tissue of the mouse was collected, fixed and then sectioned for Safranine O-Fast Green staining.
3. Results showed that the thylakoid metabolic system could effectively inhibit the progression of osteoarthritis by systematically regulating the metabolism of the chondrocyte, as shown in FIG. 6(a ) to FIG. 6(d).

The biological materials for delivering the specific cell-targeted metabolic system obtained in Embodiments 2 and 3 were respectively subjected to an uptake experiment of mixed cell culture system, an uptake experiment of encapsulation with different cell membranes, quantitative measurement of key metabolic molecule, fluorescent quantitation of substance metabolism, combined transcriptomic and metabonomic analysis of systematic metabolism regulation, and tissue section staining of in-vivo therapeutic effect, and the results were similar to those of the encapsulation of the thylakoid with the chondrocyte membrane by the filter-membrane microporous gradient extrusion method for treating osteoarthritis through intra-articular injection in Embodiment 1, which indicated that the preparation and application of the biological 1 material for delivering the specific cell-targeted metabolic system could be realized through other metabolic system components, other specific cell membranes and other encapsulating processes mentioned above.

The above are only the preferred embodiments of the present invention, and it should be pointed out that, although the present invention has been described in detail with reference to the preferred embodiments above, those skilled in the art should understand that several improvements and decorations may also be made without departing from the principle of the present invention, and these improvements and decorations should also be regarded as falling within the scope of protection of the present invention without departing from the scope defined by the claims of the present invention.

We claim:

1. A biological material for delivering a specific cell-targeted metabolic system, wherein the biological material is composed of a cell membrane of a target cell for delivery and a metabolic system component encapsulated inside.

2. The biological material for delivering the specific cell-targeted metabolic system according to claim 1, wherein the cell membrane of the target cell for delivery comprises a cell membrane of a motor system-related cell, a circulatory system-related cell, a digestive system-related cell, a urinary system-related cell, a nervous system-related cell, a germ cell, an endocrine cell and a tumor cell; the cell membrane of the motor system-related cell comprises a cell membrane of a chondrocyte, a myocyte, an osteoblast, an osteoclast and a mesenchymal stem cells the cell membrane of the circulatory system-related cell comprises a cell membrane of a hematopoietic stem cell, a monocyte, a granulocyte, a macrophage, a B lymphocyte, a T lymphocyte, an erythrocyte, a platelet, a myocardial cell and a vascular endothelial cell; the cell membrane of the digestive system-related cell comprises a cell membrane of a hepatocyte, a gastrointestinal epithelial cell, a goblet cell and an islet cell; the cell membrane of the urinary system-related cell comprises a cell membrane of a respiratory system cell such as an alveolar cell and a tracheal epithelial cell, a glomerular endothelial cell and a renal tubular epithelial cell; and the cell membrane of the nervous system-related cell comprises a cell membrane of a neuronal cell, an astrocyte, an oligodendrocyte and a microglial cell.

3. The biological material for delivering the specific cell-targeted metabolic system according to claim 1, wherein the cell membrane of the target cell for delivery is a cell membrane of a chondrocyte, a myocyte, a monocyte, a macrophage, an endothelial cell and an epithelial cell.

4. The biological material for delivering the specific cell-targeted metabolic system according to claim 1, wherein the cell membrane of the target cell for delivery is a cell membrane of a chondrocyte having definite differentiation characteristics and targeting by intra-articular injection to avoid systemic influence.

5. The biological material for delivering the specific cell-targeted metabolic system according to claim 1, wherein the metabolic system comprises an entirety and partial contents of a nucleus, a cytoplasmic matrix, a mitochondrion, a chloroplast, an endoplasmic reticulum and a Golgi apparatus.

6. The biological material for delivering the specific cell-targeted metabolic system according to claim 1, wherein the metabolic system is content component-containing thylakoid vesicle in chloroplast.

7. A preparation method for the biological material for delivering the specific cell-targeted metabolic system according to claim 1, comprising the following steps of:
extracting metabolic system vesicle to be delivered for granulation, extracting the cell membrane of the target cell for delivery, and encapsulating the metabolic system into the cell membrane, so as to obtain cell membrane vesicle encapsulating the metabolic system component.

8. The preparation method for the biological material for delivering the specific cell-targeted metabolic system according to claim 7, wherein the metabolic system vesicle to be delivered is thylakoid vesicle, which is extracted in the following mode:
a plant green leaf material and a cold buffer A are mixed by a blender in a ratio of 1 g:1 mL; the obtained solution is pressed to pass through a fine mesh cotton fabric, and a filtrate is centrifuged at 3000 g for 10 minutes; a precipitate is resuspended in a buffer B; the solution is added to 80/40% Percoll gradient solution; a part containing a green layer is collected to obtain a thylakoid; ingredients of the buffer A are sorbitol, HEPES-KOH with pH of 7.6, MgCl₂ and 0. 1% BSA; and ingredients of the buffer Bare sorbitol, HEPES-KOH with pH of 7.6, MgCl₂, EDTA and L-sodium ascorbate.

9. The preparation method for the biological material for delivering the specific cell-targeted metabolic system according to claim 7, wherein a granulating process is implemented by an ultrasonic extrusion method, which is carried out in the following mode:
the metabolic system vesicle to be delivered is subjected to an ultrasonic treatment in a bath ultrasonic instrument, and repeatedly extruded by a polycarbonate porous membrane; then the solution is centrifuged at 3000 g for 10 minutes; and a precipitate is resuspended in a buffer D, wherein ingredients of the buffer D are HEPES-KOH, MgCl₂ and sodium ascorbate.

10. The preparation method for the biological material for delivering the specific cell-targeted metabolic system according to claim 9, wherein conditions of the ultrasonic treatment of the ultrasonic instrument comprise: a No. 2 amplitude-change pole, 20% to 60% power, turning on for 2 seconds, turning off for 3 seconds, and working for 2 minutes.

11. The preparation method for the biological material for delivering the specific cell-targeted metabolic system according to claim 10, wherein a pore size of the polycarbonate membrane above is 50 nm to 200 nm.

12. The preparation method for the biological material for delivering the specific cell-targeted metabolic system according to claim 9, wherein the extraction of the cell membrane of the target cell for delivery is carried out in the following mode:
the cell is collected and resuspended in a buffer E at 4° C., repeatedly whipped with an insulin needle for 20 times to lyse the cell, and mixed with a 1 M sucrose buffer E solution in a ratio of 3:1 into a 0.25 M sucrose buffer E solution, the solution is centrifuged at 2000 g for 10 minutes, a supernatant is taken and centrifuged at 3000 g for 30 minutes, and a precipitate is the cell membrane; ingredients of the buffer E are mannitol, sucrose, Tris, MgCl₂, KCl, PMSF, a protease inhibitor without EDTA, DNase and RNase, and a pH value of the buffer E is 7.4; and a concentration of the Tris is 5 mM to 30 mM, and a concentration of the MgCl₂ is 1 mM to 20 mM.

13. The preparation method for the biological material for delivering the specific cell-targeted metabolic system according to claim 12, wherein the concentration of the Tris is 10 mM.

14. The preparation method for the biological material for delivering the specific cell-targeted metabolic system according to claim 12, wherein the concentration of the MgCl₂ is 1 mM.

15. The preparation method for the biological material for delivering the specific cell-targeted metabolic system according to claim 9, wherein an encapsulating process is carried out in the following mode:
the metabolic system component is loaded into the cell membrane vesicle by methods comprising microporous extrusion, ultrasonic hydration and a microfluidic technology.

16. The preparation method for the biological material for delivering the specific cell-targeted metabolic system according to claim 15, wherein the encapsulating process is implemented by a filter-membrane microporous gradient extrusion method, and pore sizes of filter-membrane micropores are 1000 nm, 400 nm and 200 nm gradiently.

17. An application of a biological material for delivering a specific cell-targeted metabolic system, wherein the biological material for delivering the specific cell-targeted metabolic system is applied to a target cell, so that the target cell ingests the biological material and internalizes the metabolic system, thus realizing a specific function of the metabolic system in the cell.

18. An application of a biological material for delivering a specific cell-targeted metabolic system, wherein an applying process comprises adding the biological material for delivering the specific cell-targeted metabolic system into a culture medium in an in-vitro cell culture system, and conveying the biological material for delivering the specific cell-targeted metabolic system to a specific cell through local injection, intravenous injection and the like in in-vivo use.
